(19) United States
(12) Patent Application Publication
(10) Pub. No.: US 2001/0044111 A1
(43) Pub. Date: Nov. 22, 2001

(54) Method for generating recombinant DNA molecules in complex mixtures

(76) Inventors: Brian Carr, Fremont, CA (US); Takashi Yamamoto, Fremont, CA (US); Phillip A. Patten, Mountain View, CA (US)

Correspondence Address: LAW OFFICES OF JONATHAN ALAN QUINE, P O BOX 458, ALAMEDA, CA, 94501

(21) Appl. No.: 09/808,731

(22) Filed: Mar. 14, 2001

Related U.S. Application Data
(60) Provisional application No. 60/190,774, filed on Mar. 20, 2000.

(51) Int. Cl. C12Q 1/68; G01N 33/53; C12P 19/34; C12N 5/00

(52) U.S. Cl. 435/6; 435/7.1; 435/91.1; 435/325

(57) ABSTRACT

Methods for generating libraries of recombinant DNA molecules by simultaneously recombining populations of DNA fragments in complex mixtures without cloning are provided. DNA derived from environmental samples, crude lysates, or partially enriched fractions of bacterial, or eukaryotic cells, or to cDNAs corresponding to RNA populations so derived, are fragmented and recombined. Optionally, the recombination is recursive. Methods for recovering and identifying recombinant DNA molecules encoding proteins with desirable characteristics are provided. Libraries of recombinant DNA molecules generated by the methods of the invention, as well as, cells incorporating such recombinant DNA molecules are provided.

### CROSS REFERENCE TO RELATED APPLICATION

This application claims priority to and benefit of related U.S. Provisional Application No. 60/190,774, filed Mar. 20, 2000, the disclosure of which is incorporated herein in its entirety for all purposes.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

The application of protein engineering has become widespread in generating modified proteins with desirable properties. Two significant, and contrasting, approaches have emerged. The first relies on the targeted alteration of a gene encoding a protein of interest. In this approach, structural information, gained by techniques such as fluorescence resonance energy transfer spectroscopy, NMR and X-ray crystallography of purified proteins and related family members, is used to rationally design specific sequence alterations in the gene encoding the protein. For example, binding of substrates, ligands, cofactors or ions can be refined or altered through specific amino acid substitutions that affect the conformation of the binding site. Such refinements, or alterations, are intentionally engineered through the mutation of single nucleotide substitutions giving rise to the desired amino acid alteration. An example is provided by Bonagura et al. (1999) “Conversion of an engineered potassium-binding site into a calcium-selective site in cytochrome c peroxidase.” J Biol Chem 274:37827. Based on x-ray crystallography data of a cytochrome p K(+) binding mutant, structural modeling was utilized to design specific amino acid alterations in the cation-binding loop which altered the substrate specificity from K⁺ to Ca²⁺.

This approach, while powerful, is dependent on extensive sequence and structural data predicated on the cloning of the underlying gene, and expression and purification of the encoded protein. Currently, it is difficult, in most cases, to accurately predict functional changes from structural data. In addition, rational design of mutagenesis strategies has the inherent drawback; that by use of predicative data, one excludes other possible alterations that may lead to the desired property.

An alternative approach to the use of structural data involves mutagenesis. In contrast to the ‘rational design’ approach described above, mutagenesis schemes are designed to be as random as experimental design allows. There are many advantages to this random approach. First, it does not require the existence of abundant structural data, and ideally does not require the need for assumptions about the mutations that may affect the desired property. For example, cassette mutagenesis replaces a segment of a polynucleotide sequence encoding a protein with a randomized, or partially randomized synthetic oligonucleotide. However, this technique is limited by both the size of the segment being replaced, and by the number of random sequences successfully inserted in its place. Like the previous rational design techniques, it requires that the target sequence be isolated and purified, and that the domain, e.g., a binding site, or catalytic domain, be localized within the sequence of the gene.

Linker/scanner mutagenesis techniques, while less dependent on sequence information, are of limited use in generating functional alterations with desirable effects. In such techniques, a sequence mutation is inserted at frequent intervals in a polynucleotide sequence, and the effect of the insertion is assessed. This can provide a useful first step in identifying functional domains, but is generally of little use in acquiring desirable functional alterations in the protein.

Mutagenesis by error prone polymerase chain reactions (PCR), is useful for generating random mutations in a cloned sequence. However, the proportion of deleterious mutations is high and the likelihood of recovering beneficial mutations decreases with the size of the gene. While this approach is at least theoretically applicable to uncloned sequences, or mixtures of template molecules, published protocols have suffered from a low processivity of the polymerase, making it unsuitable for application to most genes (Caldwell et al. (1992) PCR Methods and Applications 2: 28).

Thus, methods that could efficiently produce desirable alterations in proteins encoded by uncloned and uncharacterized DNA sequences are of significant value. The present invention provides methods for generating desirable functional alterations in proteins without prior knowledge of the gene sequence. In the present invention, complex mixtures, including genomic and cellular cDNA are recombined, e.g., shuffled, in vitro prior to cloning or isolation of specific genes, making it possible to recover beneficial mutant sequences based on the functional properties of the proteins they encode. These and other advantages of the invention will become apparent upon complete review of the following.

### SUMMARY OF THE INVENTION

Numerous recombination procedures, including DNA shuffling, have been widely utilized to generate diversity in nucleic acids. The present invention extends existing technology to the recombination, e.g., shuffling, of complex populations of DNA prior to amplification, cloning, or characterization of the source DNA. Accordingly, the methods and reactions provided by the invention can be used to harvest diversity from uncloned genes, uncharacterized or poorly characterized genes, and interrupted or truncated genes, among many others.

The invention provides methods for generating libraries of recombinant DNA molecules by recombining a complex population of DNA fragments, optionally in a recursive fashion. In general, the complexity of the DNA corresponds to the complexity of a genome, or, e.g., to the cellular RNAs representing the expression products of a genome. DNA fragments are derived from environmental samples, such as plants, animals, fungi, or soil or water samples, or from artificial sources such as laboratory cultures. Samples or cultures can contain prokaryotic organisms, such as bacteria, or eukaryotic organisms such as yeast, fungus or small multicellular organisms. Alternatively, the DNA is derived from cell or tissue samples of eukaryotic organisms such as a fungi, plants or animals, or the like, including humans, or from viruses grown under artificial or natural conditions. Optionally, the libraries generated by the methods of the invention are inserted into a vector, optionally including, e.g., a virus, a plasmid, a binary vector system, a cosmid, an artificial chromosome or the like.

In some embodiments, heterogeneous DNA fragments comprising homologous members are recombined. These heterogeneous DNA fragments are derived alternatively from one or multiple species or strains.

In some embodiments, the DNA fragments provided as the substrate of a recombination reaction are present in a crude cell extract. In other embodiments, the DNA is subjected to one or more enrichment processes, such as a gradient, a pulse field gel or a field inversion gel, prior to fragmenting. Virtually any method that reduces the overall size of the DNA molecules while maintaining its basic sequence integrity can be used to generate the DNA fragments. In exemplary embodiments, fragmenting the DNA sample is performed by DNAse digestion, restriction enzyme digestion, sonication, chemical shearing, mechanical shearing, primer extension, random primer extension, or the like.

The complex population of DNA fragments is then recombined. In some embodiments, the DNA fragments are recombined in a polymerase chain reaction (PCR). In an embodiment, the PCR is a primeness PCR. In some embodiments, the PCR is supplemented with a DNA molecule of interest, e.g., an isolated DNA sequence, a cloned DNA sequence, a synthetic DNA sequence, an amplified DNA sequence, or the like.

In some embodiments, one or more recombinant DNA molecules are recovered from the library. In some cases, the recovery occurs by a PCR. Primers used in the PCR can bind either to a coding or non-coding region of the recombinant DNA molecule and optionally can be partially or wholly degenerate. In one embodiment, no two primers included in the PCR anneal to a single component of the population of naturally occurring or cDNA molecules provided as recombination substrates. Thus, the parental naturally occurring or cDNA molecules cannot serve as PCR substrates. In an alternative embodiment, the primers anneal to a repetitive element such as an IS sequence, a transposon, a retrotransposon, a highly repetitive element, a middle repetitive element, an Alu sequence, a LINE sequence, a SINE sequence, or the like.

Another aspect of the invention provides for the identification of recombinant DNA molecules with desired properties from the libraries of the invention. Recombinant DNA molecules with desired properties can be identified by in vitro or in vivo screening methods or both. In some cases the screening methods are selection methods.

The invention further provides for PCR methods and reaction mixtures for generating recombinant DNA molecules from complex mixtures of DNA molecules. The complex mixtures of DNA optionally contain populations of naturally occurring DNAs and/or cDNAs. In an embodiment, primers that include linkers are used to amplify recombinant DNA molecules. In some embodiments, more than one sub-population of heterogeneous homologous DNA fragments is extended simultaneously to generate a plurality of double stranded recombinant DNA molecules. In preferred embodiments, the polymerase utilized in the PCR reaction is a thermostable DNA polymerase.

PCR mixtures containing complex populations of naturally occurring or cDNA molecules and/or recombinant DNA molecules generated therefrom are also a feature of the invention.

Libraries of recombinant DNA molecules generated by the methods of the invention, optionally inserted into vectors, are a feature of the invention, as are recombinant DNA molecules having desired properties identified by the methods of the invention.

Cells comprising recombinant DNA molecules identified according to the methods of the invention are an aspect of the invention. Such cells can be bacteria, fungus, or plant or animal cells. Additionally, transgenic organisms made by regenerating plant or animal cells bearing the recombinant DNA molecules of the invention are a feature of the invention.

### BRIEF DESCRIPTION OF THE FIGURES

FIG. 1. A schematic illustration of an exemplary method for generating recombinant DNA molecules in complex mixtures without cloning.

### DETAILED DESCRIPTION

Definitions

Unless defined otherwise, all technical and scientific terms used herein have the meaning commonly understood by a person skilled in the art to which this invention belongs. A “naturally occurring” DNA molecule is a DNA molecule that exists in the same state as it exists in nature; that is, the DNA molecule is not isolated, recombinant, or cloned.

A “complementary DNA” or “cDNA” is a DNA molecule that is complementary and colinear with an RNA molecule.

The “complexity” of a population of DNA is a measure of the amount of unique sequence in a DNA sample. Typically, the higher the complexity, the greater the number of genes present in a sample, and incidentally, the larger the genome (e.g., a gene: 10³; a bacterial genome: 10⁶; a human genome: 10⁹). Thus, in the context of the present invention, a “complex” population of DNA fragments is one with many different unique components, or “members.”

A population of DNA fragments is “heterogeneous” either because it includes members that are different from each other, (e.g., because they are derived from different species or strains), or because it is composed of different genes, including different genes of the same gene family. “Homologous members” of a population of DNA fragments are members that are related by sequence to a common ancestral gene (e.g., are members of the same gene family), and thus, share sequence similarity. “Orthologous” refers to the same gene in different species or strains. For example, interferons are a multigene family present in many species of multicellular eukaryotes, e.g., birds, mammals. Interferons can be subdivided based on structural and genetic similarity into alpha, beta, gamma, tau, etc. As descendents of a common ancestral gene, such genes are properly referred to as homologous. Nonetheless, as non-identical sequences, they are heterogeneous. The gamma interferon genes of mouse and of man, for example, are orthologous.

An “environmental sample” is a collection of cells and the material found with the cells in a natural setting. An environmental sample includes, for example, a soil sample, a water sample, a sample of a fungus, plant or animal found in nature.

A cell extract, e.g., from an environmental sample or laboratory culture, is a “crude cell extract” if it has not been subject to purification steps in addition to lysis and centrifugation of the cells. “Enrichment” of the cell extract refers to the partial purification of one or more components in the cell extract.

Introduction

The methods of the present invention utilize a complex source of uncloned DNA as the substrate for DNA recombination, e.g., shuffling, procedures. Such a source can be genomic DNA isolated or enriched from a bacterial or eukaryotic source. Alternatively, crude cell lysates containing chromosomal as well as episomal DNA provides the substrate for recombination, e.g., shuffling reactions. In some embodiments, the DNA source is complementary DNA (cDNA) corresponding to cellular RNAs. The source DNA is then fragmented, by chemical, enzymatic or mechanical means and recombined to produce novel nucleotide sequences. The recombinant DNA molecules produced by the recombination reaction(s) are globally or selectively amplified, and cloned into an expression vector. After transformation into a suitable host, recombinant DNA molecules encoding proteins or polynucleotides of interest with desirable or improved characteristics are identified by various selection or screening protocols.

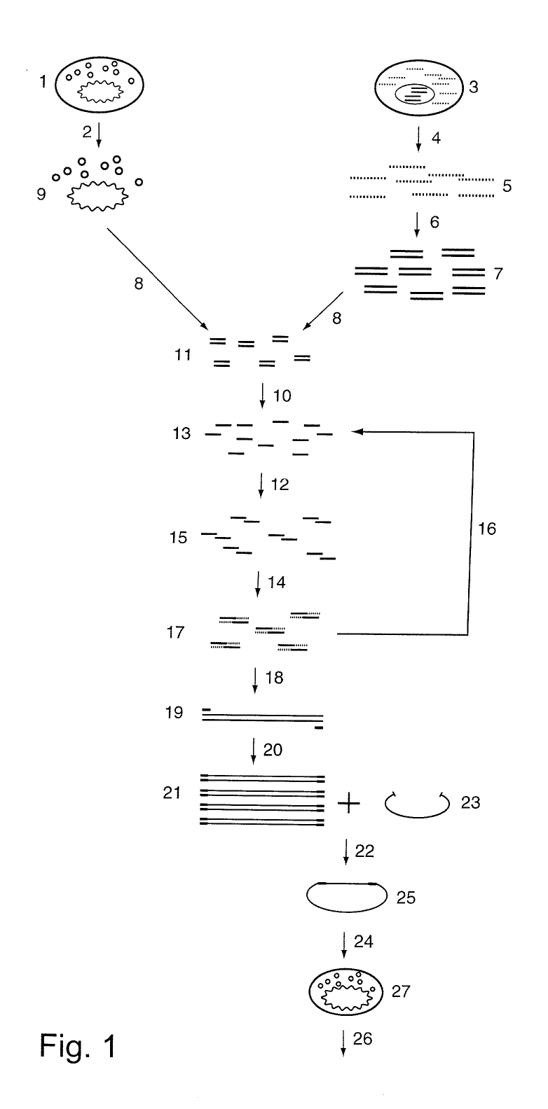

An exemplary method is illustrated schematically in FIG. 1. Briefly, a source of a complex mixture of DNA, such as one or more bacterial [1] or eukaryotic [3] cell, is lysed [2] to produce a cellular lysate [9] containing a mixture of DNA substrates. Alternatively, cellular RNAs [5] are isolated [4] and the corresponding cDNAs [7] are synthesized [6]. The DNA substrates are combined in vitro and fragmented [8]. The DNA fragments [11] are then recursively recombined in vitro by a) melting [10] the double stranded DNA fragments to generate single stranded DNA fragments [13]; b) annealing [12] the single stranded DNA fragments at regions of partial overlap [15]; c) extending [14] the partially overlapping fragments to generate a population of recombinant DNA fragments [17], and d) repeating [16] the recombination process of steps a) through d) one or more times, resulting in the production of recombinant DNA molecules comprising full length genes [19]. Because the methods of the invention involve recombination of DNA at regions of homology, related or homologous DNA sequences will anneal even in the presence of large excesses of extraneous, non-homologous, DNA fragments. Following recombination, primers that are hybridize specifically to a sequence within a gene of interest, or alternatively, that hybridize to e.g., repetitive sequences within or outside a gene of interest are annealed [18] to the recombinant DNA molecules, or some fraction thereof. One, a few, several or many of the recombinant DNA molecules in the mixture are then amplified [20] using PCR, LCR or other available methods. The amplified recombinant DNA molecules [21] are, optionally, ligated [22] into a vector [23], and transduced [24] into appropriate host cells [25] and screened [26] for desirable properties.

The following application details methods for generating novel recombinant polynucleotides encoding proteins or polypeptides with desirable characteristics without any prior cloning step. A complex DNA mixture containing numerous related and unrelated polynucleotide sequences is simultaneously recombined based on homology relationships. DNA mixtures containing the entire genomic complement of chromosomal and episomal DNA molecules is fragmented using enzymatic, chemical, or mechanical means, and recombined, for example, by DNA shuffling techniques to generate novel DNA molecules, e.g., genes, encoding products with novel and desirable characteristics. In some instances, crude cellular lysates are used. Alternatively, a sub-portion of the genomic complement is enriched based on physical characteristics such as size, density, hybridization parameters or the like. In other cases, cDNA populations corresponding to a cellular RNA complement is used as the substrate for the recombination, e.g., shuffling, reaction.

The fragmented DNA in the complex mixture is simultaneously recombined at regions of sequence similarity, e.g., based on homology relationships, by any one or a combination of a variety of DNA shuffling and other techniques. Optionally, such procedures are performed recursively. Recombinant DNA molecules so generated are then recovered, cloned into a suitable expression vector, and introduced into a host chosen based on the specific application. Recombinant DNA molecules encoding proteins or polypeptides with novel and/or desirable characteristics are then identified by selection or screening.

DNA Substrates

The present invention relates to the recombination of complex populations of DNA. The complexity of a population of DNA molecules is a measure of the unique sequence present in a sample. Typically, complexity is measured indirectly, by parameters such as hybridization, although direct sequencing provides a more definitive measure. For example, the size of an average gene is in the range of 1-10 kilobase, or 1000-10,000 base pairs, yielding a complexity measurement of 10³-10⁴ base pairs (bp). A bacterial genome typically has a complexity of ranging from about 10⁶ bp to 5×10⁷, while a eukaryotic genomes are generally larger and more complex, ranging from 10⁸ in the case of C. elegans to about 3×10⁹ for humans. While this increase in complexity generally corresponds to an overall increase in genomic size, more importantly, these complexity measurements reflect an increase in unique sequences corresponding to the regulatory and protein encoding portion of the genome.

The methods of the invention relate to the recombination of DNA sequences present in a complex mixture, e.g., a prokaryotic or eukaryotic genome, or the cDNA population corresponding to the expression products of a eukaryotic cell. In particular, sources of DNA that include many genes are particularly suitable as substrates for the methods of the invention. For example, the entire DNA complement of a population of bacteria can be provided as the substrate for the recombination, e.g., shuffling, reactions of the present invention. The DNA content of a bacterium typically contains a large circular “chromosomal” DNA molecule and a variable number of extrachromosomal “plasmid” DNA molecules which vary in size from a few to more than one hundred kilobases (kb) in size. The present invention provides methods for recombining, e.g., shuffling, these various genomic components simultaneously in a complex mixture, such as a crude cellular extract, an environmental sample or the like.

Numerous methods and variations thereon for the preparation of cellular extracts are known in the art (see, e.g., Berger, Sambrook and Ausubel, supra). In the present invention, the DNA of a population of cells, for example, one or more species or strains of bacteria are prepared, combined in vitro, and fragmented. While the methods of the present invention presume that the DNA from the various bacterial species or strains is combined and fragmented, it is of little importance whether the bacterial DNA is prepared before or after the bacterial cells have been combined, or indeed, whether the DNA is fragmented before or after it is combined in vitro. Thus, cells can be combined, lysed, and the DNA prepared and fragmented; or the cells can be lysed, the DNAs prepared and combined prior to fragmentation; or the cells can be lysed, the DNAs prepared, fragmented and subsequently combined. Which order is followed is a matter of convenience and can vary from situation to situation.

The source of cells or other materials (e.g., viruses, tissues, etc.) utilized in the production of an extract is dependent on the specific application and the material available. In some cases, cultured cells, e.g., bacterial cells grown in nutrient broth under laboratory conditions; mammalian cells maintained as adherent or suspension cultures in medium (including cell types and strains available from public domain culture collections) are a desirable source of DNA. Alternatively, environmental samples, such as soil or water samples containing microorganisms (or other small soil borne or aquatic organisms or viruses) are utilized. In other cases, tissue samples of eukaryotic cells, e.g., mammalian or human organ samples or other animal, fungal or plant tissues are the DNA source of choice. In such cases, cDNA is optionally prepared from cellular RNA. The methods elected for the preparation of the corresponding DNA sample depend on the choice of material, and whether genomic or cDNA samples are preferred. Appropriate choices and methods can be selected by one skilled in the art.

In some situations, it is preferable to perform the recombination, e.g., shuffling, reaction in a crude cell extract containing the DNA. For example, some bacterial species, (or strains) grow poorly under laboratory conditions, making it difficult to obtain DNA from cultured populations. In these cases, the DNA can be collected from an environmental sample. Rather than purifying the DNA following lysis, the DNA is fragmented in a crude extract, with or without boiling (e.g., incubation at 95° C. for 15 minutes) to inactivate nucleases and other enzymes that can interfere with subsequent manipulations.

For example, an environmental soil sample can be collected and added to a suitable quantity of a buffered solution to produce a suspension, e.g., of bacterial cells and soil particles. The soil particles, and other insoluble inorganic materials can be removed by sedimentation or filtration, and the cells collected from the suspension by centrifugation. After recovery, the bacterial cells are lysed according to methods known in the art (see e.g., Berger, Kimmel and Ausubel) to produce a crude cell extract. The DNA in the crude extract is then fragmented and the fragments provided as substrates in the reactions of the invention.

Similarly, genomic DNA from eukaryotic cells can be employed as described above. Some eukaryotic microorganisms, such as yeast, as well as some multicellular eukaryotes, e.g., Caenorhabditis elegans, have compact genomes, with little or no intervening or repetitive DNA sequences interrupting the mRNA encoding regions. Thus genomic DNA from such organisms can be used directly.

Alternatively, it is desirable to subject the nucleic acids present in the crude cell extracts to some sort of preparative procedure. Many such procedures for the preparation of cellular DNA and RNA from bacterial and eukaryotic cells are well-known in the art, and many such procedures and reagents are available as kits (e.g., cesium chloride centrifugation; PEG precipitation; QIAGEN™ columns (www.qiagen.com); TRIzol™ (www.lifetech .com), etc., see, e.g., Berger, Sambrook and Ausubel, as well as individual manufacturers).

In some cases, DNA preparations are subjected to one or more enrichment steps, e.g., density or other gradient separation, pulsed field electrophoresis, field inversion electrophoresis, etc., prior to recombination, e.g., by DNA shuffling. For example, if the target sequence is known to be present on a plasmid in a bacterial cell, cesium chloride gradient centrifugation, pulsed field gel electrophoresis or field inversion gel electrophoresis can be used to enrich for plasmid DNA. In the case of a mammalian, or other eukaryotic, gene that is mapped to a particular chromosome pulse field gel electrophoresis or other methods, can be used to enrich the sample for the desired chromosome prior to fragmentation. Unique sequences enriched from DNA of “higher” eukaryotes by differential rehybridization (Cot) analysis can also be employed advantageously.

In other eukaryotic organisms, where a significant proportion of the genome is composed of intervening and repetitive sequences, it is desirable, even in the absence of cloning, to enrich for relevant, or coding, regions of the genome. One alternative approach is to selectively remove DNA comprising repetitive elements. This is readily accomplished by fragmenting or partially fragmenting the substrate DNA, melting it and allowing it to rehybridize. The more highly repetitive the DNA, the more quickly it rehybridizes. For a given sample of DNA, the rehybridization curve, or so-called Cot curve can be established, and the rapidly annealing double stranded DNAs (repetitive DNAs) removed by binding to hydroxylapatite columns by techniques known in the art (see, e.g., Berger, Sambrook and Ausubel). Alternatively, cellular RNA corresponding to the genes expressed in a particular cell or tissue type is isolated, and corresponding cDNAs are synthesized. Either approach preserves the complexity, or at least that portion of the overall complexity that is relevant to the function of the particular cell or tissue type, while reducing bias inherent in samples containing a large proportion of repetitive DNA.

While the foregoing discussion has focused on cellular sources of DNA, e.g., bacterial or eukaryotic cells, viral genomes are also amenable to the methods of the present invention. Viral genomes can be isolated from cultures or from an environmental source such as infected cells. Techniques for isolating viruses and recovering their DNA, or for recovering RNA genomes and producing corresponding cDNAs are well established in the art.

Fragmentation of Nucleic Acids

A number of approaches can be used to produce “fragmented” nucleic acids. Fragmented nucleic acids can be provided by mechanically shearing nucleic acids, by enzymatically or chemically cleaving nucleic acids, by partially synthesizing nucleic acids, by random primer extending or directed primer extending nucleic acids, by incorporating cleavable elements into the nucleic acids during synthesis, or the like. Templates or starting materials for such procedures include the DNA and RNA nucleic acids of the invention, e.g., genomic DNAs, cDNAs, mRNAs, nRNAs, cloned nucleic acids, cloned DNAs, cloned RNAs, plasmid DNAs, viral DNAs, viral RNAs, artificial chromosome DNAs, cosmid DNAs, branched DNAs, in vitro amplified nucleic acids, PCR amplified nucleic acids, LCR amplified nucleic acids, SDA nucleic acids, Qβ-replicase amplified nucleic acids, nucleic acid sequence-based amplified (NASBA) nucleic acids, transcription-mediated amplified (TMA) nucleic acids, oligonucleotides, nucleic acid fragments, restriction fragments, combinations thereof and any other available nucleic acid. Nucleic acids can be unpurified, or partially or substantially purified prior to fragmentation.

For example, nucleic acids can be fragmented enzymatically, e.g., using a DNAse. An appropriate concentration and incubation time are determined empirically to result in fragments of the desired length, (e.g., 50-500 bp). Alternatively, immobilized DNAse on support resin beads can be used for fragmentation, with DNA to be fragmented passing over a column made of the beads. This avoids a potential problem of contaminating salts in the DNA solution (e.g., a crude cell extract) which are removed by gel filtration. An extension of this procedure is to encapsulate the DNAse in a polymeric (plastic) resin. Wang et al. (1997) “Biocatalytic plastics as active and stable materials for biotransformations” Nat Biotechnol 2:15:789 and the references therein describe biocatalytic plastic technology generally. Resin encapsulation has the advantage of stabilizing the enzyme greatly: no loss of activity is seen even after 30 or more days. Synthesis of a stable DNAse resin avoids the need to re-calibrate the column to account for loss of activity. Using a fixed initial concentration of DNA, DNA fragment size can be determined by the flow rate through the column. Fractions can be collected containing known fragment sizes suitable for DNA recombination reactions, e.g., DNA shuffling reactions.

Other means of enzymatic digestion include partial or complete digestion with one or more restriction enzymes. For example, overlapping DNA fragments can be generated by partial digestion of a sample with one or more enzymes, either sequentially or in a single reaction. If multiple reactions are performed sequentially, the restriction enzyme(s) are optionally heat inactivated or removed by extraction in organic solvents, e.g., phenol, chloroform. Overlapping fragments can also be generated by dividing a sample into fractions which are, independently, partially or completely, fragmented with different restriction enzymes that generate different fragmentation patterns, i.e., that cut the DNA at different sites. Following digestion, the fractions are recombined, thus, providing overlapping fragments.

Alternatively, nucleic acids are mechanically sheared, e.g., by vortexing, sonicating, point-sink shearing or other similar operations. Mechanical shearing of nucleic acids has the advantage of being sequence independent, which, at times is desirable, e.g., where no bias is desired in the sheared nucleic acid fragments. For example, the point-sink shearing method is described in Thorstenson et al. (1998) “An automated hydrodynamic process for controlled, unbiased DNA shearing,: Genome Research 8:848:855. Although this method typically generated relatively large DNA fragments (500-1000 bp), the size of fragments can be reduced by increasing the velocity of the solution, decreasing the size of the channel, vibrating the channel or the like.

Alternatively, in some applications, e.g., second or subsequent rounds of recombination with the nucleic acids of the invention. DNA fragmentation is achieved via incorporation of cleavage targets into nucleic acids of interest. Modified nucleotides or other structures are incorporated into nucleic acids during synthesis of the nucleic acids. These modified nucleotides or other structures become cleavage points within a nucleic acid into which they are incorporated. One example of this approach is described, e.g., in PCT US96/19256. As noted in the '256 application, nucleic acid synthesis can be conducted to produce nucleic acids of interest (e.g., via PCR or synthetic methods), incorporating uracil into the nucleotides in a stochastic or directed fashion. The PCR products are then fragmented by digestion with two enzymes, a Uracil N-glycosylase (UNG) and an AP endonuclease, e.g., Endonuclease IV (End) which form strand breaks at the uracil residues. A fundamental advantage of Ung-End fragmentation is that fragmentation is simply a function of uracil content.

Similarly, RNA nucleotides can be incorporated into DNA chains (synthetically or via enzymatic incorporation); these nucleotides then serve as targets for cleavage via RNA endonucleases. A variety of other cleavable residues are known, including certain residues which are targets for enzymes or other residues and which serve as cleavage points in response to light, heat or the like. Where polymerases are currently not available with activity permitting incorporation of a desired cleavage target, such polymerases can be produced using, e.g., current shuffling methods to modify the activity of existing polymerases, or to acquire new polymerase activities.

Simple chain termination methods can also be used to produce nucleic acid fragments, e.g., by incorporating dideoxy nucleotides into the reaction mixture(s) of interest.

Nucleic Acid Diversification

Following digestion or shearing of the source DNA into fragments, the DNA fragments are recombined to produce novel, recombinant DNA molecules. Optionally, the recombination is performed recursively. For example, nucleic acids, e.g., comprising genes encoding polypeptides or proteins of interest, can be recombined in vitro by any of a variety of techniques, e.g., as discussed in the references below, including e.g., DNAse digestion of nucleic acids to be recombined followed by ligation and/or PCR reassembly of the nucleic acids. The present invention extends previously described methods in the following important respects. The present invention utilizes complex mixtures of uncloned, e.g., naturally occurring, DNA as a substrate. Without any prior amplification or cloning steps, the complex source DNA is fragmented and recombined, e.g., shuffled, in vitro.

For example, DNA corresponding to the entire genomic complement of a bacterial cell, or from multiple strains or species of bacteria can be procured, either as crude cell lysates, or as partially or substantially enriched (i.e., purified) fractions. The DNA substrate, whether it is the product of a single species or strain, or of many combined species or strains, is fragmented using a method suitable for the procured DNA. Considerations involved in the selection of an appropriate method include the quantity of available DNA, available equipment, contaminants present in the DNA mixture or solution. As a general principle, the more contaminating compounds (e.g., salts) present in the mixture, the more suitable are mechanical methods, e.g., sonication, trituration. In contrast, the more pure, or devoid of inhibitory compounds, the more suitable are enzymatic techniques such as DNAse digestion. The method most appropriate to a particular application will be readily apparent to one of skill in the art.

The DNA fragments are then recombined in vitro. Briefly, the DNA fragments are melted (by temperature or chemical means) and allowed to anneal based on regions of sequence similarity. Because the regions of sequence similarity are short relative to the overall length of the DNA fragments in the reaction mixture, annealing occurs between similar, but non-identical, DNA fragments. Typically, annealing occurs between homologous gene sequences, but heterologous or divergent genes can be trapped by short regions of sequence similarity, thus, enhancing the diversity generated by the recombination process. In the complex mixtures of the present invention, it is often desirable to perform the annealing for a longer period of time than in “conventional” polymerase mediated recombination procedures. In addition, increasing the DNA concentration, and/or increasing the salt concentration can be used to favor homology based annealing of nucleic acids in the context of the present invention.

A DNA polymerase, for example, a thermostable DNA polymerase, then extends the annealed fragments resulting in recombination among the various DNA fragments in the reaction mixture. Successive cycles of melting, annealing and extension, most typically performed by PCR result in the assembly of substantially full-length recombinant gene sequences. In some cases, it is advantageous to remove the polymerase activity after each cycle of extension, prior to the next annealing step. This can be accomplished, for example, by utilizing a heat sensitive polymerase (e.g., Klenow, DNA Poll holoenzyme) and heat inactivating the enzyme between successive cycles of annealing and extension. Alternatively, the polymerase can be removed by extraction with organic solvents, e.g., phenol, chloroform, isoamyl alcohol, or a mixture thereof. A polymerase having a Stag” (His tag, epitope tag, etc.) can be removed between cycles by passage over an appropriate binding column, e.g., Ni-NTA agarose, etc.

The present methods provide for the recombination, e.g., shuffling of complex mixtures of uncloned DNA, making it possible to recombine DNA from environmental samples, and bulk cultures of cells, without the prior cloning of gene sequences. This offers the significant advantage of providing the diversity inherent in a population of organisms (of the same or different species) without the loss of sequence variation inherent in the cloning process.

Like the in vitro, in vivo, and whole cell shuffling procedures outlined below, and described in detail in the cited references, the methods of the present invention utilize regions of sequence similarity to mediate recombination. Because these processes involve regions of sequence similarity (e.g., homology), like DNAs anneal even in the presence of large excesses of non-homologous DNA. In addition, small regions of sequence similarity present in unrelated or non-homologous genes are trapped in the annealing process, further adding to the diversity of the recombination, e.g., shuffling, mixture.

Because the DNA of each contributing cell, (e.g., each species, strain or isolate) is digested to very small sizes (e.g., 20-500 bp), reassembly of parental genes is no more favored than during conventional nucleic acid recombination procedures, such as DNA shuffling. The ratio of reconstituted parental genes to recombinant genes depends on the similarity between the genes and the conditions of the reaction, not upon the presence of non-homologous DNA.

It will further be appreciated that sequences across the genome are recombined, e.g., shuffled, simultaneously based on their homology relationships. Thus, multiple gene families are “independently” and simultaneously recombined during the reactions of the invention. This feature offers the additional benefit of providing the substrates for optimizing a multi-step pathway in a single screening procedure without prior knowledge of the genes required to establish the pathway.

Recovery of Recombinant DNA Molecules

Following one or more rounds of diversification, e.g., by DNA shuffling, the recombinant DNA molecules derived from the genomic or complementary DNA fragments are recovered by amplification and cloning. Amplification of recombinant DNA molecules encoding full-length polypeptides of interest can be accomplished in two theoretically and practically distinct manners. Firstly, recombinant molecules corresponding to the entire population can be recovered using non-specific primers in a polymerase chain reaction. Alternatively, specific primers corresponding to a subset of the recombinant molecules can be utilized.

Non-specific primers include primers that anneal to repetitive or semi-repetitive DNA sequences, such as bacterial IS sequences, transposon sequences, retrotransposon sequences, and middle/highly repetitive elements in eukaryotic DNA, e.g., Alu sequences, LINE sequences, SINE sequences, etc. For a detailed list of transposable and repetitive elements, see, for example, Berg and Howe, eds., (1989) Mobile DNA ASM, Washington, D.C.; Shapiro, ed. (1983) Mobile Genetic Elements Academic Press, New York; and Sheratt, ed. (1995) Mobile Genetic Elements Oxford Press, New York, and references therein.

Using this approach, a complex library of amplified DNA, including, e.g., shuffled DNA sequences of interest, is generated. The library of amplified sequences can be screened for a property inherent in the polynucleotides, e.g., hybridization to a known sequence or sequences. Alternatively, the amplified sequences can be cloned into an expression vector, introduced into an appropriate host and screened for a desired property encoded by the polynucleotide, e.g., enzymatic activity. To facilitate cloning, primers incorporating linkers, or restriction enzyme recognition sites can be employed. This approach is of particular use when sequence information is unavailable or incomplete. In such cases, recombinant DNA molecules encoding polypeptides with a desired property can, nonetheless, be recovered on the basis of one or more functional assay used to assess the desired property.

In cases where sequence information corresponding to a family of sequences is available, specific primers can be selected to meet any of a number of criteria. For example, primers corresponding to a single known family member can be used to amplify all members of the family, including recombinant, e.g., shuffled, members that include the primer binding sequences. Alternatively, primer pairs can be designed to recover only recombinant molecules, and to avoid parental molecules by selecting highly specific primers that anneal to sequences on two different parental DNA molecules. While this approach calls for increased sequence information, it results in the enrichment of the library for desirable sequences and, thus, simplifies the screening or selection process. Many variations lying between these two extremes, such as partially or wholly degenerate primers, can be envisioned without changing the basic principle of the invention.

Although, for purposes of discussion, diversification (e.g., by DNA shuffling) and recovery have been demarcated, it will be clear to one of skill in the art that this distinction is largely artificial. Recombination strategies that simultaneously enrich for the recovery of recombinant molecules of interest from complex DNA mixtures are favorably employed in the context of the present invention. For example, in one combinatorial approach, a sample consisting of a genomic or cellular cDNA population, is divided into multiple pools. In a first round, each pool is denatured and annealed to a specific or degenerate oligonucleotide primer, optionally containing a convenient restriction site to facilitate subsequent manipulations. Optionally, the primer is linked to a solid support. The primer is extended, and the double stranded products, all of which share at least minimal regions of sequence similarity with the primer, are recovered. In the case of a support-linked oligonucleotide, unbound components of the mixture can be washed away. Alternatively, methods are well established for separating single stranded from double stranded DNA molecules. In subsequent rounds of denaturing, annealing and extension, the pools are combined and redivided to allow recombination between novel pairs of DNA strands.

Numerous methods for the amplification of rare, as well as abundant, DNA molecules from complex populations are available, and well known to those of skill in the art. Examples of techniques sufficient to direct persons of skill through in vitro amplification methods, including the polymerase chain reaction (PCR) the ligase chain reaction (LCR), Qβ-replicase amplification and other RNA polymerase mediated techniques (e.g., NASBA), e.g., for the production of the homologous nucleic acids of the invention are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369: 684-685 and the references therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase.

Recovery of recombinant DNA molecules with desired properties can be increased by combining the methods of the present invention, for example, with any of the described shuffling procedures or other methods for increasing diversity. Such variations include (a) recombination, e.g., shuffling, of only positive clones recovered (low-diversity shuffling), (b) recombination, e.g., shuffling, of positive clones with the parental DNA (high-diversity shuffling), (c) recombination, e.g., shuffling, of positive clones with other members of homologous or related gene families (family shuffling), especially, e.g., where added functionality is desirable (e.g., in providing enzymes with unique functions such as the ability to catalyze multi-step reaction pathways) (d) spiking the recombination, e.g., shuffling, reaction with oligos encoding, e.g., particular catalytic or other structural domains, (e) serial passage of recombinant, e.g., shuffled, clones through an E. coli mutator strain (e.g. E. coli mutD5), (f) recombination, e.g., shuffling, of clones derived from a library selected on the basis of functional properties, or (g) any combination of the above.

Complementary Techniques

Numerous methods for generating molecular diversity can be practiced in conjunction with the methods of the present invention. These methods can be practiced separately, and/or in combination either as an adjunct to the methods of the invention, e.g., by supplementing or “spiking” the reactions mixtures with nucleic acids corresponding to those described and produced by the following methods, or in subsequent rounds of recombination utilizing the nucleic acids, e.g., libraries of recombinant DNA molecules, of the invention.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, which can be selected or screened for nucleic acids that encode proteins with or which confer desirable properties. Following diversification by one or more of the methods herein, or otherwise available to one of skill, any nucleic acids that are produced can be selected for a desired activity or property. This can include identifying any activity that can be detected, for example, in an automated or automatable format, by any of the assays in the art, as described in further detail below. A variety of related (or even unrelated) properties can be evaluated, in series or in parallel, at the discretion of the practitioner.

Descriptions of a variety of diversity generating procedures suitable for producing modified nucleic acid sequences which can be used in conjunction with the methods of the present invention, are found in the following publications and the references cited therein. These methods provide a departure point for the methods of the present invention. Soong, N. et al. (2000) “Molecular breeding of viruses” Nat Genet 25(4):436-439; Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wild-type cassettes” BioTechniques 18:194-195; Stemmer et al., (1995) “Single-step assembly of a gene and entire plasmid form large numbers of oligodeoxy-ribonucleotides” Gene, 164:4953; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382403), deletion mutagenesis (Eghtedarzadeh & Henikoff (1986) “Use of oligonucleotides to generate large deletions” Nucl. Acids Res. 14: 5115), restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundström et al. (1985) “Oligonucleotide-directed mutagenesis by microscale ‘shot-gun’ gene synthesis” Nucl. Acids Res. 13: 3305-3316), double-strand break repair (Mandecki (1986) “Oligonucleotide-directed double-strand break repair in plasmids of Escherichia coli: a method for site-specific mutagenesis” Proc. Natl. Acad. Sci. USA, 83:7177-7181; and Arnold (1993) “Protein engineering for unusual environments” Current Opinion in Biotechnology 4:450-455). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection;” WO 00/00632, “Methods for Generating Highly Diverse Libraries;” WO 00/09679, “Methods for Obtaining in Vitro Recombined Polynucleotide Sequence Banks and Resulting Sequences;” WO 98/42832 by Arnold et al., “Recombination of Polynucleotide Sequences Using Random or Defined Primers;” WO 99/29902 by Arnold et al., “Method for Creating Polynucleotide and Polypeptide Sequences;” WO 98/41653 by Vind, “An in Vitro Method for Construction of a DNA Library;” WO 98/41622 by Borchert et al., “Method for Constructing a Library Using DNA Shuffling;” WO 98/42727 by Pati and Zarling, “Sequence Alterations using Homologous Recombination;” WO 00/18906 by Patten et al., “Shuffling of Codon-Altered Genes;” WO 00/04190 by del Cardayre et al. “Evolution of Whole Cells and Organisms by Recursive Recombination;” WO 00/42561 by Crameri et al., “Oligonucleotide Mediated Nucleic Acid Recombination;” WO 00/42559 by Selifonov and Stemmer “Methods of Populating Data Structures for Use in Evolutionary Simulations;” WO 00/42560 by Selifonov et al., “Methods for Making Character Strings, Polynucleotides & Polypeptides Having Desired Characteristics;” and PCT/US00/26708 by Welch et al., “Use of Codon-Varied Oligonucleotide Synthesis for Synthetic Shuffling.”

In addition, details regarding certain diversity generating methods are found in U.S. patent application “SINGLE-STRANDED NUCLEIC ACID TEMPLATE-MEDIATED RECOMBINATION AND NUCLEIC ACID FRAGMENT ISOLATION” by Affholter, filed Sep. 6, 2000 (U.S. Ser. No. 09/656,549).

In brief, several different general classes of sequence modification methods, such as mutation, recombination, etc., are applicable to the present invention and are set forth, e.g., in the references above. The following exemplify some of the different types of formats for diversity generation that can be employed in combination with the methods of the present invention, e.g., for further diversifying recombinant nucleic acids generated using the methods of the invention.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants is described in several of the references above, e.g., in Stemmer (1994) Proc. Natl. Acad. Sci. USA 91:10747-10751.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above.

Whole genome recombination methods can also be used in which whole genomes of cells or other organisms are recombined, optionally including spiking of the genomic recombination mixtures with desired library components (e.g., recombinant nucleic acids recovered according to the methods of the present invention). These methods have many applications, including those in which the identity of a target gene is not known. Details on such methods are found, e.g., in WO 98/31837 by del Cardayre et al. “Evolution of Whole Cells and Organisms by Recursive Sequence Recombination;” and in, e.g., WO 00/04190 by del Cardayre et al., also entitled “Evolution of Whole Cells and Organisms by Recursive Recombination.”

Synthetic recombination methods can also be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., WO 00/42561 by Crameri et al., “Oligonucleotide Mediated Nucleic Acid Recombination;” PCT/US00/26708 by Welch et al., “Use of Codon-Varied Oligonucleotide Synthesis for Synthetic Shuffling;” WO 00/42560 by Selifonov et al., “Methods for Making Character Strings, Polynucleotides & Polypeptides Having Desired Characteristics;” and WO 00/42559 by Selifonov and Stemmer “Methods of Populating Data Structures for Use in Evolutionary Simulations.”

In silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis/gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in WO 00/42560 by Selifonov et al., “Methods for Making Character Strings, Polynucleotides & Polypeptides Having Desired Characteristics” and WO 00/42559 by Selifonov and Stemmer “Methods of Populating Data Structures for Use in Evolutionary Simulations.” Extensive details regarding in silico recombination methods are found in these applications.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “SINGLE-STRANDED NUCLEIC ACID TEMPLATE-MEDIATED RECOMBINATION AND NUCLEIC ACID FRAGMENT ISOLATION” by Affholter, U.S. Ser. No. 09/656,549, filed Sep. 6, 2000.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombination procedure termed “incremental truncation for the creation of hybrid enzymes” (“ITCHY”) described in Ostermeier et al. (1999) “A combinatorial approach to hybrid enzymes independent of DNA homology” Nature Biotech 17:1205. This approach can be used to generate an initial a library of variants which can optionally serve as a substrate for one or more in vitro or in vivo recombination methods. See, also, Ostermeier et al. (1999) “Combinatorial Protein Engineering by Incremental Truncation,” Proc. Natl. Acad. Sci. USA, 96: 3562-67; Ostermeier et al. (1999), “Incremental Truncation as a Strategy in the Engineering of Novel Biocatalysts,” Biological and Medicinal Chemistry, 7: 2139-44.

Mutational methods which result in the alteration of individual nucleotides or groups of contiguous or non-contiguous nucleotides can be favorably employed to introduce nucleotide diversity into recombinant nucleic acids produced according to the methods of the invention. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave & Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g. a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson et al. (1998) U.S. Pat. No. 5,783,431 “Methods For Generating and Screening Novel Metabolic Pathways,” and Thompson, et al. (1998) U.S. Pat. No. 5,824,485 “Methods for Generating and Screening Novel Metabolic Pathways”) and their use to identify protein activities of interest has been proposed (In addition to the references noted above, see, Short (1999) U.S. Pat. No. 5,958,672 “Protein Activity Screening of Clones Having DNA from Uncultivated Microorganisms”). Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215: 471) prior to diversifying according to any of the methods described herein.

Libraries can be biased towards nucleic acids which encode proteins with desirable enzyme activities. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in Short (1999) U.S. Pat. No. 5,939,250 for “Production of Enzymes Having Desired Activities by Mutagenesis.” Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods be applied to the present invention as well. Random or semi-random mutagenesis using doped or degenerate oligonucleotides is also described in, e.g., Arkin and Youvan (1992) “Optimizing nucleotide mixtures to encode specific subsets of amino acids for semi-random mutagenesis” Biotechnology 10:297-300; Reidhaar-Olson et al. (1991) “Random mutagenesis of protein sequences using oligonucleotide cassettes” Methods Enzymol. 208:564-86; Lim and Sauer (1991) “The role of internal packing interactions in determining the structure and stability of a protein” J. Mol. Biol. 219:359-76; Breyer and Sauer (1989) “Mutational analysis of the fine specificity of binding of monoclonal antibody 51F to lambda repressor” J. Biol. Chem. 264:13355-60); and “Walk-Through Mutagenesis” (Crea, R; U.S. Pat. Nos. 5,830,650 and 5,798,208, and EP Patent 0527809 B1.

It will readily be appreciated that any of the above described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides.

A recombinant nucleic acid produced by recursively recombining one or more polynucleotide of the invention with one or more additional nucleic acid also forms a part of the invention. The one or more additional nucleic acid may include another polynucleotide of the invention; optionally, alternatively, or in addition, the one or more additional nucleic acid can include, e.g., a nucleic acid encoding a naturally-occurring protein or polypeptide, or a subsequence thereof, or any homologous sequence or subsequence thereof, (e.g., as found in Genbank or other available literature, or newly identified), or, e.g., any other homologous or non-homologous nucleic acid (certain recombination formats noted above, notably those performed synthetically or in silico, do not require homology for recombination).

Also included in the invention is a cell containing any resulting recombinant nucleic acid, nucleic acid libraries produced by recursive recombination of the nucleic acids set forth herein, and populations of cells, vectors, viruses, plasmids or the like comprising the library or comprising any recombinant nucleic acid resulting from recombination (or recursive recombination) of a nucleic acid as set forth herein with another such nucleic acid, or an additional nucleic acid.

After amplification of the recombinant DNA molecules, recovery is accomplished by cloning the amplified sequence into a vector, for example, a cloning vector or an expression vector. The vector can be, e.g., in the form of a plasmid, a cosmid, an artificial chromosome or a virus. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See Giliman and Smith (1979) Gene 8:81; Roberts et al. (1987) Nature 328:731; Schneider et al. (1995) Protein Expr. Purif. 6435:10; Ausubel, Sambrook, Berger (all supra). A catalogue of Bacteria and Bacteriophages useful for cloning is provided, e.g., by the ATCC: Gherna et al. (eds) (1992) The ATCC Catalogue of Bacteria and Bacteriophage. Additional basic procedures for sequencing, cloning and other aspects of molecular biology and underlying theoretical considerations are also found in Watson et al. (1992) Recombinant DNA Second Edition, Scientific American Books, NY.

Following cloning into a suitable vector, the recombinant DNA molecules of the invention can be transduced into host cells by standard methods including electroporation, infection by viral vectors, microinjection, Calcium phosphate precipitation, PEG mediated transfection, high velocity ballistic penetration by small particles with the nucleic acid either within the matrix of small beads or particles, or on the surface or any other technique known in the art for the transduction of nucleic acids into the host cell of choice. For example, additional methods suitable for the transduction of plant cells include use of pollen as vector (WO 85/01856), or use of Agrobacterium tumefaciens or A. rhizogenes carrying a T-DNA plasmid in which DNA fragments are cloned.

Alternatively, recovery is accomplished by directly integrating the recombinant DNA into a bacterial or eukaryotic cell without prior insertion into a vector. For example, primers that incorporate sequence corresponding to a unique region of a genome (e.g., a yeast, Saccharomyces cerevisiae, chromosome) are ligated onto the ends of a generated recombinant PCR product. These PCR products can be directly transformed into yeast, and selected for integration by insertion into a gene for which selection is possible. For example one can insert into a metabolic gene, such as, HIS4 or LEU2, and screen for auxotrophy. Similarly, the recombinant, e.g., shuffled, products can be ligated to an antibiotic resistance gene, or DNA containing promoter, enhancer, or other cis-acting element required for expression in yeast or other eukaryotic cell. Homology can also be introduced on the ends of a PCR product by ligating larger regions corresponding to a target gene or region into which insertion is desired onto the ends of PCR products digested with restriction enzymes. In this manner, large DNA regions, on the order on 1 kb per side or more, can be added to the ends of PCR products. DNA added to the ends of PCR products then mediates integration by homologous recombination at high frequency. This method also works for prokaryotic organisms which undergo obligate homologous recombination. This method is applicable to other eukaryotes, including plants which are capable of integrating exogenous DNAs by homologous recombination, albeit at lower frequency. Thus, the resulting recombinant, e.g., shuffled, products do not necessarily require cloning into vectors.

The engineered host cells can be cultured in conventional nutrient media modified as appropriate for such activities as, for example, activating promoters or selecting transformants.

Screening is then performed to identify recombinant DNA molecules that encode polypeptides with desired properties. Any screening or selection method known in the art is applicable to the present invention and choice is determined by the particular property desired.

Generation of Transgenic Cells and Organisms

The present invention also relates to host cells and organisms which are transformed with the nucleic acids of the invention, and the production of polypeptides of the invention, by recombinant techniques. Host cells are genetically engineered (i.e., transformed, transduced or transfected) with the vectors of this invention, which may be, for example, a cloning vector or an expression vector. The vector may be, for example, in the form of a plasmid, a viral particle, a phage, etc. The engineered host cells can be cultured in conventional nutrient media modified as appropriate for such activities as, for example, activating promoters or selecting transformants. The culture conditions, such as temperature, pH and the like, are those previously used with the host cell selected for expression, and will be apparent to those skilled in the art and in the references cited herein, including, e.g., Freshney (1994) Culture of Animal Cells, a Manual of Basic Technique, third edition, Wiley-Liss, New York and the references cited therein. A variety of cell culture media are described in Atlas and Parks (eds) The Handbook of Microbiological Media (1993) CRC Press, Boca Raton, Fla. (Atlas). Additional information for plant cell culture is found in available commercial literature such as the Life Science Research Cell Culture Catalogue (1998) from Sigma-Aldrich, Inc (St Louis, Mo.) (Sigma-LSRCCC) and, e.g., the Plant Culture Catalogue and supplement (1997) also from Sigma-Aldrich, Inc (St Louis, Mo.) (Sigma-PCCS). Additional details regarding plant cell culture are found in R. R. D. Croy, Ed. (1993) Plant Molecular Biology Bios Scientific Publishers, Oxford, U.K.

The present invention also relates to the production of transgenic organisms, which may be bacteria, yeast, fungi, plants or animals. A thorough discussion of techniques relevant to bacteria, unicellular eukaryotes and cell culture may be found in references enumerated above and are briefly outlined as follows. Several well-known methods of introducing target nucleic acids into bacterial cells are available, any of which may be used in the present invention. These include: fusion of the recipient cells with protoplasts (e.g., bacterial, fungal, yeast or plant protoplasts or spheroplasts) containing the DNA, electroporation, lipofection, projectile bombardment, and infection with viral vectors (discussed further, below), etc. Bacterial cells can be used to amplify the number of plasmids containing DNA constructs of this invention. The bacteria are grown to log phase and the plasmids within the bacteria can be isolated by a variety of methods known in the art (see, for instance, Sambrook). In addition, a plethora of kits are commercially available for the purification of plasmids from bacteria. For their proper use, follow the manufacturer's instructions (see, for example, EasyPrep™, FlexiPrep™, both from Pharmacia Biotech; StrataClean™, from Stratagene; and, QIAprep™ from Qiagen). The isolated and purified plasmids are then further manipulated to produce other plasmids, or used to transfect cells of other species, including eukaryotic species.

General texts which describe molecular biological techniques useful herein, including the use of vectors, promoters and many other relevant topics related to, e.g., the preparation of DNA samples from bacterial and eukaryotic cells, and the cloning and expression of bacterial and eukaryotic genes, include Berger and Kimmel, Guide to Molecular Cloning Techniques, Methods in Enzymology volume 152 Academic Press, Inc., San Diego, Calif. (“Berger”); Sambrook et al., Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)).

While a thorough discussion of techniques relevant to bacteria, unicellular eukaryotes and cell culture may be found in references enumerated above, additional techniques valuable in the production of transgenic animals also include, e.g., Hogan et. al., Manipulating the Mouse Embryo, second edition, (1994) Cold Spring Harbor Press, Plainview.

Techniques for transforming plant cells with nucleic acids are generally available and can be adapted to the invention by the introduction of nucleic acids encoding recombinases, fusion proteins and evolved proteins. In addition to Berger, Ausubel and Sambrook, useful general references for plant cell cloning, culture and regeneration include Jones (ed) (1995) Plant Gene Transfer and Expression Protocols—Methods in Molecular Biology, Volume 49 Humana Press Towata N.J.; Payne et al. (1992) Plant Cell and Tissue Culture in Liquid Systems John Wiley & Sons, Inc. New York, N.Y. (Payne); and Gamborg and Phillips (eds) (1995) Plant Cell, Tissue and Organ Culture; Fundamental Methods Springer Lab Manual, Springer-Verlag (Berlin Heidelberg New York) (Gamborg).

Kits

The present invention also provides a kit or system for performing one or more of the reactions, e.g., a shuffling reaction for the production of a recombinant DNA library, described herein. The kit or system can optionally include a set of instructions for practicing one or more of the methods described herein; one or more assay components that optionally include at least one recombinant, isolated and/or artificially evolved enzyme or at least one cell that includes one or more such enzymes or both, and one or more reagents; and a container for packaging the set of instructions and the assay components. The assay component can optionally include at least one immobilized enzyme as described above, or at least one such enzyme free in solution, or both.

Recombinant, isolated, or artificially evolved nucleic acids, or the proteins or peptides they encode, or a combination thereof, can be supplied as assay components of the kits or systems of the present invention. In a further aspect, the present invention provides for the use of any component or kit herein, for the practice of any method or assay herein, and/or for the use of any apparatus or kit to practice any assay or method herein.

### EXAMPLES

Evolution of Novel Bacillus Thuringiensis δ Endotoxins

The present invention provides methods for producing novel proteins with desirable properties without the requirement that the nucleic acids encoding the protein, or its precursor, be cloned, isolated or even known. The following example is focused on a known class of genes/gene products to simplify discussion. However, it will be appreciated that the methodology is analogous for the recovery of previously undescribed sequences.

The bacterium Bacillus thuringiensis produces proteins, often referred to as δ endotoxins, with insecticidal properties. These bacterial endotoxins have proven of widespread interest, particularly in the realm of agriculture, due to their protective properties against certain insect species (e.g., Lepidoptera spp. such as Plutella xylostella, Spodoptera frugiperda, Spodoptera exigua, Heliothis virescens, Trichoplusia ni, Coleoptera spp. such as Leptinotarsa decemlineatea, as well as Diptera spp., etc.). For review, see, e.g., Schnepf et al. (1998) Microbiology and Molecular Reviews 62:775. Endotoxins that protect against a broader range of insects, insects with limited sensitivity to current endotoxins, insects resistant to current endotoxins, or fungal parasites, for example, are of intense interest. Such endotoxins, as well as endotoxins with other desirable properties, can be produced by the methods of the present invention.

For example, various B. thuringiensis strains are available from public domain cell culture repositories, e.g., the ATCC, Bethesda, Md. Cells comprising one or several of these strains are acquired for immediate use, or grown in culture to sufficient numbers for the specific application. The bacteria are then concentrated by sedimentation or centrifugation, if necessary, and lysed, e.g., by digestion with lysozyme and proteinase K (see, e.g., Berger, Sambrook, Ausubel, all supra) to generate a crude cell extract. Alternatively, samples of B. thuringiensis are isolated directly from soil. The samples are treated to discard gross contaminants, such as rock and soil particles, and to concentrate the bacterial cells for further processing as described for cultured strains. Alternatively, microbiological or physical methods are used to either select, screen, or enrich for Bacillus thuringiensis isolates from soil samples. For example, Bacillus thuringiensis may be identified from crude platings of soil bacteria using a selective medium such as an acetate selection medium (see, e.g., Travers et al. (1987) “Selective Process for efficient isolation of soil Bacillus spp.,” in Applied and Environmental Microbiology 53:1263. It will be appreciated that bacterial cells derived from one or a combination of these means are appropriate sources of DNA for subsequent manipulation.

The crude cell extract containing the entire genomic complement, including chromosomal and plasmid DNAs is then aliquoted. As previously indicated, multiple strains or samples can be processed independently to this point, or combined prior to lysis at the discretion of the practitioner. The DNA in the lysate is then fragmented, for example by sonication. Alternatively, DNAse or restriction enzyme digestion can be employed to fragment the DNA, either prior to or following boiling to inactivate endogenous proteases, nucleases and the like. Again, it is unimportant whether individual samples corresponding to individual strains or samples are combined prior to or following fragmentation of their component DNA.

The DNA fragments are then recombined, e.g., recursively recombined, in vitro, as previously described in the references enumerated above. Briefly, the random duplex DNA fragments are denatured, then allowed to reanneal on the basis of, typically short, regions of sequence similarity (or homology). A polymerase is employed to extend the partially overlapping fragments to generate duplex DNA molecules. This process is repeated, with extended members reannealing with new counterparts in each subsequent cycle, until a diverse population of recombinant DNA molecules is generated. As this process occurs in a complex mixture corresponding to the genomic complement of a bacterial strain, or strains, many genes, and families of genes are simultaneously recombined on the basis of their homology relationships. This is of particular interest, as small regions of sequence similarity “trap” diversity from dissimilar genes in the recombination process.

Following the recombination, e.g., shuffling, process, the recombinant DNA is rescued by amplification using a polymerase chain reaction and cloning. For example, numerous insertion sequences (IS) are known to reside throughout bacterial genomes, including the genome of B. thuringiensis. PCR primers corresponding to IS231 can be used to amplify recombinant sequences across the bacterial genome. The amplified sequences constituting a library of recombinant B. thuringiensis sequences are then inserted into a vector of choice for transformation into a host cell suitable for the subsequent screening of the recombinant DNA molecules. For example, after amplification the ends of the amplified products can be filled in or digested with a restriction enzyme (either naturally occurring or engineered into the primer) and cloned into a plasmid such as Bluescript (Stratagene: www.stratagene.com) containing an inducible or constitutive promoter for regulating transcription of the inserted amplification product.

The plasmids incorporating the recombinant DNA molecules of the invention are then transformed into a host suitable for screening, e.g., functional attributes of the proteins encoded by the recombinant DNA molecules. For example, in the δ-endotoxin case, individual or pooled colonies representing individual library members can be grown, and extracts produced and assayed for insecticidal activity.

The following exemplary procedure is one such favorable method for generating recombinant δ-endotoxins. Whole cellular genomic DNA from the HD1 strain of B. thuringiensis is prepared by standard lysozyme, proteinase K digestion procedures. The HD1 strain is particularly suited to the methods of the invention as it carries two endogenous δ-endotoxin genes on a naturally occurring plasmid. The prepared DNA is then divided into two aliquots for fragmentation. One aliquot is digested with the restriction enzyme BsaAI, which cuts predominantly in the latter third of the endotoxin gene. The other aliquot is digested with AseI, which yields a different restriction pattern. It will be obvious to one of skill in the art that other restriction enzymes are also suitable for the purpose of generating fragments, and appropriate substitutions can be determined by the practitioner. Following digestion, the restriction enzymes are heat inactivated. The two aliquots are then combined, providing overlapping nucleic acid fragments. The combined sample is heated to denature the fragments, and annealed at 60° C. for an extended period of time, e.g., from 8-16 hours, or longer, in a buffered solution containing 100 mM NaCl. The annealed fragments are then extended with a DNA polymerase, e.g., Klenow, DNA Poll holoenzyme, or Taq polymerase, at an appropriate incubation temperature and time selected dependent on the chosen enzyme. The resulting full-length, or substantially full length, recombinant δ-endotoxin genes are then recovered by amplification in a standard PCR, using primers that are designed to preferentially amplify recombinant products.

The example just discussed relies on in vitro recombination, e.g., shuffling methods to produce novel protein products. However, in vivo methods can readily be applied in conjunction with the in vitro methods described above. For example, the various strains of B. thuringiensis can be combined in vivo by protoplast fusion techniques established in the art, see, e.g., Schaefer and Hotchkiss, (1978) “Fusion of Bacterial Protoplasts” in Methods in Cell Biology, Prescott, ed. pp 149-158, ASM, New York; Kennett (1979) Methods in Enzymology, 58:345. The fused protoplasts are rescued, then the DNA is prepared and fragmented as described above.

Generation of Novel Interferon-like Molecules

The methods of the present invention are equally suited to the generation and isolation of novel proteins from multicellular eukaryotes, including higher plants, and animals such as mammals. For such applications, it is preferred to utilize cDNA (complementary DNA) rather than genomic DNA as the starting material. As previously indicated, the genomic DNA of many multicellular eukaryotes is interspersed with intervening and repetitive elements of many varieties. Frequently, one or more intervening sequence, i.e., introns, interrupts the coding regions of a gene. In some instances, non-coding and intervening sequences can extend a gene as much as ten times or greater, the length of its coding sequence.

Currently, PCR methods are conveniently applied to sequences of one to several kilobases or less. Larger regions are amplified less efficiently, with greater error, and are subject to biases based on sequence and secondary structure. While maintaining a high degree of complexity, cDNA of many multicellular eukaryotes offers the significant advantage of being devoid, or at least reduced, of much of the non-coding sequence present in many eukaryotic genes. This facilitates the recovery of novel coding sequences from complex mixtures by PCR methods readily available in the art.

For example, the method of the present invention can be utilized to isolate, e.g., novel interferon-like molecules. Cells from a tissue of interest, e.g., peripheral blood, fibroblasts, etc., are lysed and total RNA or mRNA is recovered by methods known in the art (see, e.g., Berger, Ausubel and Sambrook). Numerous kits and reagents are available (e.g., Triazol; www.lifetech.com.; RNeasy; www.qiagen.com.) and can be utilized according the manufacturer's instructions to simplify such procedures. Cells can be human cells, or other mammalian cells, or non-mammalian cells, known or not known to contain interferon-like molecules. A first strand cDNA is then synthesized by reverse transcription using random (oligo dT, degenerate, or specific) primers, e.g., using a commercially available kit (from Qiagen: RNeasy, Ambion: Retroscript™, among many others). The second strand is then extended to generate double stranded cDNAs. Total cDNA from such procedures is then fragmented and recombined, e.g., shuffled as previously described. Recombinant cDNAs encoding interferon-like molecules, or other desired proteins are rescued using either specific or non-specific primers and cloned into a suitable vector. The recombinant cDNAs are then introduced into host cells which are screened to identify cells which produce molecules with desirable interferon-like activities. For example, transduced cells expressing recombinant cDNAs can be incubated with cells expressing a interferon inducible reporter gene, e.g., β-galactosidase. Conversion of a chromogenic or fluorogenic substrate by induced β-galactosidase can be measured in a high-throughput format. Alternatively, antiviral or other interferon activity can be evaluated.

Production of Eukaryotic Genes Lacking Introns

Eukaryotic DNA from species such as mouse or human (as well as many plants) with a high proportion of intervening sequences can alternatively be recombined, e.g., shuffled with, and recovered in the context of genes from organisms such as S. cerevisiae, or C. elegans with compact genomes. The relatively compact genomes which lack introns or with only a few small introns can be exploited to regenerate functional minimally interrupted, or non-interrupted, genes using the methods of the present invention. For example, novel recombinases with humanized properties, e.g., substrate specificity, antigenicity, protein interaction, etc., can be produced in the following manner.

RecA homologues are present in organisms from bacteria, to yeast, to mammals, including humans. RecA homologues in mammals are interrupted by introns, while the RecA homologue, Rad51 of the yeast, S. cerevisiae, are not. DNA from yeast, human and optionally from additional organisms such as C. elegans and mouse, is fragmented and recombined, e.g., shuffled, as described above. Primers specific for, e.g., yeast Rad51, or for a combination of different RecA/Rad51 homologues, are then utilized to amplify a subset of the recombinant products. Due to the inherent size limitations of PCR under normal conditions, (i.e., under 10 kb) the products amplified are likely to be genes that lack introns, or have only small introns. The resulting recombinant products can then be cloned into an expression vector, transformed into eukaryotic cells, such as yeast or cultured mammalian cells such as HELA cells or COS cells, and assayed functionally for the desired property. For example, in a two step procedure, recombinant products can first be transformed into RecA/Rad51 deficient yeast cells and assayed for complementation. Recombinant products with recombinase activity can then be transfected into mammalian cells and further assayed for desirable functional attributes.

Alternatively, genes without introns, or with reduced introns can be produced by supplementing the assembly reaction used to reassemble DNA fragments originating from a species with numerous and/or large introns with synthetic introns. Short synthetic introns with flanking regions of homology to known intron/exon junctions are added to the reaction mix, where they are incorporated during gene reassembly, substituting for the larger, naturally occurring introns. This results in an overall reduction in the size of the gene facilitating recovery by subsequent PCR. In a similar manner, short oligonucleotides that span the exon-intron junction can be employed to create a defacto cDNA-like molecule (i.e., one in which all or some of the introns are removed).

Hybrid Prokaryotic/Eukaryotic Genes

Similarly, DNA from prokaryotic and eukaryotic sources can be fragmented and recombined, e.g., shuffled, to produce chimeric genes with desirable properties. For example, novel Type I and/or Type II polyketide synthases can be produced by combining DNA from prokaryotic and eukaryotic (e.g., fungal, plant) genomes. In the case of Type II polyketides, one can mix genomic DNA from fungi with bacterial DNA (e.g., from Streptomyces spp.), fragment, amplify, and rescue using oligonucleotides based on sequence of one of the Streptomycete polyketide synthase genes. Alternatively, one oligonucleotide corresponding to a streptomycete gene and one from a fungal gene can be employed.

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method for generating a library, the method comprising: fragmenting a complex population of naturally occurring DNA molecules or complementary DNA (cDNA) molecules in vitro to generate a population of DNA fragments; recursively recombining the complex population of DNA fragments, which recombining is homology dependent, thereby assembling at least one recombinant DNA molecule.

2. The method of claim 1, comprising simultaneously recombining DNA fragments across the complex population.

3. The method of claim 2, comprising recombining a population of heterogeneous DNA fragments comprising homologous members.

4. The method of claim 3, wherein the heterogeneous DNA fragments comprise DNA fragments from organisms of the same or different species.

5. The method of claim 1, wherein the population of DNA molecules fragmented has a complexity in excess of about 10.

6. The method of claim 5, wherein the population of DNA molecules fragmented has a complexity in excess of about 10.

7. The method of claim 1, comprising fragmenting a population of genomic DNA molecules.

8. The method of claim 7, comprising deriving the genomic DNA molecules from at least one cell.

9. The method of claim 8, comprising deriving the genomic DNA molecules from an environmental sample comprising the at least one cell.

10. The method of claim 8, comprising selecting the at least one cell from among a bacterium, a yeast cell, or a Caenorhabditis elegans cell.

11. The method of claim 10, wherein the bacterium is a Bacillus.

12. The method of claim 11, wherein the bacterium is Bacillus thuringiensis.

13. The method of claim 8, further comprising fusing a plurality of protoplasts prior to deriving the genomic DNA from at least one cell, which at least one cell is a product of the fused protoplasts.

14. The method of claim 8, comprising fragmenting the population of genomic DNA molecules in a crude cell extract.

15. The method of claim 14, further comprising boiling the crude extract prior to fragmenting the population of genomic DNA molecules.

16. The method of claim 8, further comprising performing one or more enrichment steps prior to fragmenting the population of genomic DNA molecules.

17. The method of claim 16, comprising performing the one or more enrichment step by a gradient, a pulse field gel or a field inversion gel.

18. The method of claim 1, comprising fragmenting a DNA genome or cDNA corresponding to an RNA genome of at least one virus.

19. The method of claim 1, comprising fragmenting a population of cDNA molecules.

20. The method of claim 19, comprising fragmenting a population of cDNA molecules corresponding a population of cellular RNA molecules.

21. The method of claim 20, further comprising isolating the cellular RNA molecules from a prokaryotic or eukaryotic cell.

22. The method of claim 21, wherein the eukaryotic cell is selected from among a multicellular plant or animal.

23. The method of claim 1, comprising fragmenting the complex population of DNA molecules by DNAse digestion, sonication, chemical shearing or mechanical shearing.

24. The method of claim 1, comprising recursively recombining the population of DNA fragments by at least one polymerase chain reaction.

25. The method of claim 24, wherein the at least one polymerase chain reaction is a primerless polymerase chain reaction.

26. The method of claim 24, further comprising supplementing the polymerase chain reaction with at least one DNA molecule of interest.

27. The method of claim 26, wherein the at least one DNA molecule of interest comprises a synthesized DNA molecule, an isolated DNA molecule, a cloned DNA molecule, or an amplified DNA molecule.

28. The method of claim 27, wherein the at least one DNA molecule of interest comprises a synthetic intron or an oligonucleotide that spans an intron/exon junction.

29. The method of claim 1, further comprising recovering at least one recombinant DNA molecule

30. The method of claim 29, comprising recovering the at least one recombinant DNA molecule by a polymerase chain reaction.

31. The method of claim 29, wherein the polymerase chain reaction is primed by primers which hybridize to a coding or non-coding sequence of the at least one recombinant DNA molecule.

32. The method of claim 31, wherein the primers do not hybridize to a single component of the population of naturally occurring DNA molecules or cDNA molecules.

33. The method of claim 31, wherein the primers hybridize to a repetitive DNA element.

34. The method of claim 33, wherein the repetitive DNA is located on a plasmid.

35. The method of claim 33, wherein the repetitive sequence is an IS sequence, a transposon sequence, retrotransposon sequence, a highly repetitive sequence, a middle repetitive sequence, an Alu sequence, a LINE sequence, or a SINE sequence.

36. The method of claim 31, wherein the primers are partially or wholly degenerate.

37. The method of claim 1, further comprising inserting the at least one recombinant DNA molecule recovered into a vector.

38. The method of claim 37, wherein the vector is a virus, a plasmid, a cosmid, or an artificial chromosome.

39. The method of claim 38, wherein the plasmid is an Agrobacterium plasmid.

40. The method of claim 39, wherein the Agrobacterium plasmid comprises a binary vector system.

41. The method of claim 37, further comprising identifying at least one recombinant DNA molecule with a desired property.

42. The method of claim 41, comprising identifying the at least one recombinant DNA molecule with a desired property by at least one of an in vitro or in vivo screening method.

43. The method of claim 42, wherein the screening method is a selection method.

44. A polymerase chain reaction (PCR) method, the method comprising:
(i) providing a population of DNA fragments, which DNA fragments comprise at least one of a complex population of naturally occurring DNA molecules or a complex population of cDNA molecules, in a buffered reaction mixture comprising a multiplicity of nucleotides comprising adenine, cytosine, guanine and thymine, and at least one DNA polymerase;
(ii) denaturing the population of DNA fragments;
(iii) annealing at least a sub-population of DNA fragments;
(iv) incubating the sub-population of annealed DNA fragments, which sub-population of annealed DNA fragments is present in a mixture comprising the complex population of naturally occurring DNA molecules or the complex population of cDNA molecules, such that the at least one DNA polymerase extends the sub-population of annealed DNA fragments into a plurality of double stranded recombinant DNA molecules.

45. The PCR method of claim 44, further comprising repeating steps (ii) through (iv) one or more time.

46. The PCR method of claim 44, further comprising amplifying at least one recombinant DNA molecule using one or more primers.

47. The PCR method of claim 46, wherein the one or more primer comprises a linker.

48. The PCR method of claim 44, wherein the DNA polymerase comprises a thermostable DNA polymerase.

49. A polymerase chain reaction (PCR) method, the method comprising:
(i) providing a population of DNA fragments, which DNA fragments comprise at least one of a complex population of naturally occurring DNA molecules or a complex population of cDNA molecules, in a buffered reaction mixture comprising a multiplicity of nucleotides comprising adenine, cytosine, guanine and thymine, and at least one DNA polymerase;
(ii) denaturing the population of DNA fragments;
(iii) simultaneously annealing at least two sub-populations of heterogeneous DNA fragments comprising homologous members, thereby providing a mixture of hybridized heterogeneous homologous DNA fragments;
(iv) incubating the mixture of hybridized heterogeneous homologous DNA fragments such that the at least one DNA polymerase extends the hybridized heterogeneous homologous DNA fragments into a plurality of double stranded recombinant DNA molecules.

50. The PCR method of claim 49, further comprising repeating steps (ii) through (iv) one or more time.

51. The PCR method of claim 49, further comprising amplifying at least one recombinant DNA molecule using one or more primers.

52. The PCR method of claim 51, wherein the one or more primer comprises a linker.

53. The PCR method of claim 49, wherein the DNA polymerase comprises a thermostable DNA polymerase.

54. A PCR mixture comprising a buffer; a population of DNA fragments, which DNA fragments comprise at least one of a complex population of naturally occurring DNA molecules or a complex population of cDNA molecules; and a population of recursively recombined DNA molecules, which recursively recombined DNA molecules comprise a plurality of heterogeneous non-homologous recombinant DNA molecules.

55. A library of recombinant DNA molecules produced by the method of claim 1.

56. A recombinant DNA molecule inserted into a vector produced by the method of claim 37.

57. A recombinant DNA molecule with a desired property identified by the method of claim 41.

58. A cell comprising at least one recombinant DNA molecule of claim 54.

59. The cell of claim 58, wherein the cell is a bacterium, a fungus, a plant or an animal.

60. A cell comprising at least one recombinant DNA molecule inserted into a vector of claim 56.

61. The cell of claim 60, wherein the cell is a bacterium, a fungus, a plant or an animal.

62. A cell comprising at least one recombinant DNA molecule with a desired property of claim 57.

63. The cell of claim 62, wherein the cell is a bacterium, a fungus, a plant or an animal.

64. A method of producing a transgenic organism comprising regenerating at least one plant or animal cell of claim 63.
